(19) United States
(12) Patent Application Publication
(10) Pub. No.: US 2025/0058054 A1
(43) Pub. Date: Feb. 20, 2025

(54) Sub-assembly for a medicament delivery device

(71) Applicant: SHL Medical AG, Zug (CH)

(72) Inventors: Christopher Foster, Zug (CH); Anders Boström, Nacka Strand (SE); Ming-Ting Yin, Taoyuan City (TW)

(21) Appl. No.: 18/724,276

(22) PCT Filed: Dec. 13, 2022
(86) PCT No.: PCT/EP2022/085592

§ 371 Date: Jun. 26, 2024

(30) Foreign Application Priority Data
Dec. 27, 2021 (EP) 21217875.0

(51) Int. Cl. A61M 5/315; A61M 5/20

(52) U.S. Cl. CPC A61M 5/3158; A61M 5/31571; A61M 2005/2073

(57) ABSTRACT

A The present disclosure provides a sub-assembly for a medicament delivery device configured to expel medicament from a medicament container. The sub-assembly includes a housing, a pre-tensioned plunger rod, a holding sleeve fixedly attached to the housing, the holding sleeve comprising at least one holding arm arranged to lock the plunger rod in its pre-tensioned state, and a rear cap having a blocking surface arranged to prevent the holding arm from releasing the plunger rod from its pre-tensioned state. The sub-assembly further comprises an activation button having a locking surface, the activation button being operatively arranged to, upon activation, move from a first state in which the locking surface is abutting the rear cap to prevent the movement of the holding arm from releasing the plunger rod, to a second state in which the locking surface is displaced to thereby allow the holding arm to move radially outwards to release the plunger rod

### TECHNICAL FIELD

The present disclosure generally relates to a medicament delivery device for expelling medicament from a medicament container. In particular, the present disclosure relates to a sub-assembly for a medicament delivery device.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various drawbacks do still exist in the current technology.

Many medicament delivery devices comprise components which are movable relative each other. One type of relative movement between components may activate the medicament delivery device, e.g. exposing the delivery member or initiating expel of medicament from the medicament delivery device. Another type of relative movement between components may result in that two components are secured relative each other, e.g. by axially or radially fixating one component to another, or by means of a locking element. The relative fixation of components to avoid relative movement is often desired prior to using the medicament delivery device. That is, in the default state of the medicament delivery device, relative movement between components are undesired, as such relative movement may result in an unintentional actions of the medicament delivery device. Such unintentional actions may e.g. be the 3o result of unsatisfactory relative fixation of components. In considering these problems, the applicant has appreciated that various developments could be made to help improve the medicament delivery devices on the market today, which are set out in more detail below.

### SUMMARY

An object of the present disclosure is thus to provide a medicament delivery device which solves, or at least mitigates, problems of the prior art.

There is hence according to a first aspect of the present disclosure provided a sub-assembly for a medicament delivery device configured to expel medicament from a medicament container. The sub-assembly comprises: a housing having a proximal end and a distal end; a pre-tensioned plunger rod operably arranged to, upon activation, act on the medicament container for expelling a medicament; a holding sleeve fixedly attached to the housing, the holding sleeve comprising at least one holding arm arranged to lock the plunger rod in its pre-tensioned state; a rear cap having a blocking surface arranged to prevent the holding arm from releasing the plunger rod from its pre-tensioned state by restricting movement of the holding arm, and being operatively configured to move distally from a blocking position to a non-blocking position in which the blocking surface is distant from the position restricting movement of the holding arm; wherein the sub-assembly further comprises an activation button having a locking surface, the activation button being operatively arranged to, upon activation, move from a first state in which the locking surface is abutting the rear cap to prevent the movement of the holding arm from releasing the plunger rod, to a second state in which the locking surface is displaced to thereby allow the holding arm to move radially outwards to release the plunger rod.

An effect obtainable thereby is an improved activation and release of the plunger rod. That is, both the rear cap and the activation button are operated to enable the holding arm to move radially outwards to release the plunger rod. Thus, by the configuration of the rear cap to move distally from a blocking position to a non-blocking position in which the blocking surface is distant from the position restricting movement of the holding arm, and the configuration of the activation button to move from a first state in which the locking surface is abutting the rear cap to prevent the movement of the holding arm from releasing the plunger rod, to a second state in which the locking surface is displaced to thereby allow the holding arm to move radially outwards to release the plunger rod, an improved activation and release of the plunger rod is provided. In other words, by arranging the rear cap in its non-blocking position, and the activation button in its second state, the holding arm is allowed to move radially outwards to release the plunger rod. Moreover, by such configuration, the sequential order in which the rear cap is arranged in its non-blocking position and the activation button is arranged in its second state may be varied.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to at least one example embodiment, the at least one holding arm is at least two holding arms. Thus, the holding sleeve may comprise at least two holding arms arranged to lock the plunger rod in its pre-tensioned state. According to at least one example embodiment, the at least one holding arm is at least four holding arms. Thus, the holding sleeve may comprise at least four holding arms arranged to lock the plunger rod in its pre-tensioned state.

According to at least one example embodiment, the blocking surface of the rear cap is arranged to abut a first holding arm, or a first sub-set of the at least two or four holding arms, to prevent movement of the corresponding holding arm(s) from releasing the plunger rod from its pre-tensioned state. Alternatively, or additionally, the locking surface of the activation button is arranged to abut a second holding arm, or a second sub-set of the at least two or four holding arms (different to the first holding arm, and the first sub-set of the at least two or four holding arms, respectively) to prevent the movement of the corresponding holding arm(s) from releasing the plunger rod. However, according to at least one alternative embodiment, the blocking surface of the rear cap is arranged to abut a portion of the activation button, to thereby prevent movement of the corresponding holding arm(s) from releasing the plunger rod from its pre-tensioned state. Thus, the rear cap may be arranged such that it does not limit the movement of any holding arm(s) by direct contact, but instead restrict movement of the holding arms(s) via direct contact with the activation button.

According to at least one example embodiment, the non-blocking position is a final distal position of the rear cap. Alternatively, or additionally, the blocking position may be referred to as a proximal position of the rear cap.

According to at least one example embodiment, the rear cap comprises a locking interacting surface arranged to abut the locking surface of the activation button in the first state to thereby lock the rear cap from moving to its non-blocking position.

Thus, the activation button may in addition to its interaction with the at least one holding arm, also interact with the rear cap to lock the rear cap from moving to its non-blocking position. According to at least one example embodiment, the locking interacting surface of the rear cap is comprised in a protrusion of the rear cap, or in a distally facing end surface of the rear cap. According to at least one example embodiment, the locking surface of the activation button is comprised in a protrusion of the activation button, or in a proximally facing end surface of the activation button.

According to at least one example embodiment, the locking interacting surface is arranged distant from the locking surface of the activation button in the second state to thereby enable the rear cap to move to its non-blocking position.

Thus, the activation button may interact with the rear cap to un-lock the rear cap from its blocking position, and enable the rear cap to move to its non-blocking position. That is, according to at least one example embodiment, in the second state, the activation button enables the rear cap to move to its non-blocking position, but it does not necessarily bring the rear cap into its non-blocking position. For example, the locking interacting surface may be moved relative to the locking surface when the activation button is brought from its first state into its second state. Hereby, the rear cap is un-locked from its blocking position, but necessary brought into its non-blocking position. According to at least one example embodiment, the sub-assembly is configured such that the locking surface is rotated relative to the locking interacting surface when the activation button is brought from its first state into its second state. According to at least one example embodiment, the sub-assembly is configured such that the locking surface is moved axially, or in a horizontal plane, relative to the locking interacting surface when the activation button is brought from its first state into its second state.

According to at least one example embodiment, the rear cap comprises an indentation in which a portion of the activation button is movable to thereby enable the rear cap to move distally from the blocking position to the non-blocking position independently of the activation button.

Hereby, the rear cap is configured to move from its blocking position to its non-blocking position independently of whether the activation button is its first state or second state.

According to at least one example embodiment, the blocking surface of the rear cap is arranged to abut the holding arm of the holding sleeve in the blocking position.

Hereby, the rear cap is arranged to restrict movement of the holding arm by direct contact between the blocking surface and the holding arm.

For example, the rear cap comprises a distal sleeve portion arranged to abut at least one holding arm, wherein the distal sleeve portion comprises the previously mentioned indentation. Thus, the distal sleeve portion may comprise the blocking surface of the rear cap. The indentation in the distal sleeve portion may e.g. shaped as a radial cut-out extending axially through a portion of the distal sleeve portion. The distal sleeve portion is typically the most distal portion of the rear cap. Correspondingly, the activation button may comprise at least two activation button legs extending proximally from a distal portion of the activation button. At least one of the two activation button legs is arranged to move in the axial direction within the indentation. According to at least one example embodiment, another one of the at least two activation button legs is arranged to abut at least one holding arm.

According to at least one example embodiment, the blocking surface of the rear cap is arranged to abut at least a proximal sleeve portion of the activation button in the blocking position to prevent the holding arm to release the plunger rod from its pre-tensioned state by restricting movement of the holding arm.

Thus, the proximal sleeve portion of the activation button is arranged in between the holding arm and the blocking surface of the rear cap. In other words, the blocking surface of the rear cap prevents the proximal sleeve portion of the activation button to move radially outwards in order to restrict movement of the holding arm.

According to at least one example embodiment, the activation button comprises an inner facing surface arranged to abut the holding arm of the holding sleeve in the first state.

For example, the shape of at least a portion of the activation button is tubular. Thus, the inner facing surface may be the inner surface of such tubularly shaped portion. For example, the tubularly shaped portion may extend proximally from the previously described distal portion of the activation button.

According to at least one example embodiment, the holding sleeve comprises a base portion from which the holding arm distally extends, wherein the holding arm is flexibly arranged relative to the base portion.

Hereby, the base portion of the holding sleeve may provide for mechanical stability, e.g. for the plunger rod and any spring housed therein, while the holding sleeve still provides the flexible functionality owing to the arrangement of the holding arm. As the holding sleeve may comprise more than one holding arm, each holding arm is preferably arranged to distally extend from the base portion in a flexible manner.

According to at least one example embodiment, the holding arm is arranged to move from an unflexed state in which the plunger rod is locked in its pre-tensioned state to a flexed state in which the plunger rod is released when the rear cap is arranged in its non-blocking position and the activation button is brought to its second state.

According to at least one example embodiment, a plurality of holding arms extend distally from the base portion, the holding arms being flexibly arranged relative to the base portion and configured to move from the unflexed state to the flexed state in which the plunger rod is released.

The holding arm, or each one of the holding arms, may comprise a knob or protrusion configured to interact with the plunger rod in the unflexed state. For this purpose, the plunger rod may comprise a corresponding knob, protrusion or indentation configured to interact with the knob or protrusion of the holding arm(s).

According to at least one example embodiment, the sub-assembly further comprises a delivery member cover arranged in the housing and extending proximally from the proximal end of the housing, the delivery member cover being configured to, during the medicament delivery operation, move linearly relative to the housing from a protracted position to a retracted position in which the delivery member cover is received further in the housing.

According to one embodiment, in the retracted position of the delivery member cover, a proximal end of the delivery member cover is arranged closer to the proximal end of the housing compared to in the protracted position. For example, in the retracted position, the proximal end of the delivery member cover is distant from the proximal end of the housing by a first axial distance, which first axial distance is smaller than a second axial distance between the proximal end of the delivery member cover and the proximal end of the housing in the protracted position.

According to at least one example embodiment, the delivery member cover comprises a distal end portion having a pushing surface arranged to abut the rear cap when the delivery member cover is moved to its retracted position to thereby move the rear cap to its non-blocking position.

Hereby, an efficient way of moving the rear cap from its blocking position to its non-blocking position is provided. For example, the delivery member cover is tubularly shaped, and the distal end portion arranged as a sleeve. Correspondingly, the base portion of the rear cap is tubularly shaped, and a proximal end portion thereof is arranged as a sleeve. Thus, the sleeved shaped distal end portion of the delivery member cover may be arranged to push the sleeved shaped base portion of the rear cap as the delivery member cover is moved into its retracted position.

According to at least one example embodiment, the delivery member cover is configured to house the delivery member of the medicament container in the protracted position. In the retracted position of the delivery member cover, the delivery member is exposed and ready for expelling the medicament. Thus, during a medicament delivery operation, the delivery member cover is positioned at the injection site, and upon a distally applied force, the delivery member cover is moved from its protracted position into its retracted position to expose the delivery member. Furthermore, as the delivery member cover is moved to its retracted position, the pushing surface of the distal end portion of the delivery member cover abuts the rear cap, typically the previously mentioned base portion of the rear cap, to thereby distally move the rear cap into its non-blocking position. Thereafter, by activating the activation button and thus arranging the activation button in the second state, the holding arm(s) move radially outwards as a result of the pre-tensioned plunger rod, whereupon the plunger rod is released and moved proximally within the housing to interact with the medicament container for expelling the medicament via the delivery member. However, for embodiments in which the rear cap and the delivery member cover are arranged to move independently of each other, the activation of the activation button may be performed prior to moving the delivery member cover into its retracted position. Moreover, and as will be described below, the delivery member cover may be locked in its protracted position by the activation button. For such embodiments, the activation button is first activated, whereafter the delivery member cover may be moved into its retracted position.

According to one embodiment, the holding sleeve is fixedly attached to the delivery member cover. Hereby, joint axial movement of the delivery cover member and the holding sleeve is achieved. For example, the holding sleeve is axially and rotationally fixed relative to the delivery member cover, and is thus axially movable together with the delivery member cover. The delivery member cover may be axially locked in its protracted position, subsequent to a medicament delivery operation, to safely protect the delivery member in the delivery member cover. That is, after use, the holding sleeve may be arranged in a final state being a non-blocking position, in which it is axially locked together with the delivery member cover. Thus, for such embodiments, the holding sleeve and the delivery member cover cannot move axially subsequent to the lockout operation. As the delivery member cover is axially locked, the delivery member is prevented from again being exposed.

According to at least one example embodiment, the locking surface of the activation button is a first locking surface, and the activation button further comprises a second locking surface arranged distant to the first locking surface, wherein in the first state of the activation button, the second locking surface is abutting the delivery member cover to prevent the movement of the delivery member cover into its retracted position, wherein in the second state of the activation button, the second locking surface is displaced to thereby allow the delivery member cover to move into its retracted position.

Hereby, the activation button may prevent the rear cap from moving into its non-blocking position, and additionally prevent the delivery member cover from moving into its retracted position. In other words, activation of the activation button into its second state, enables the rear cap to move into its non-blocking position, and enables the delivery member cover to move into its retracted position. For example, the activation button comprises a first radially extending protrusion comprising the first locking surface, and a second radially extending protrusion comprising the second locking surface. Correspondingly, the rear cap may comprise a first structure comprising a first locking interacting surface (e.g. the previously mentioned locking interacting surface) and a first indentation engageable with the first radially extending protrusion in a lockable manner. Moreover, the delivery member cover may comprise a second locking interacting surface and a second indentation engageable with the second radially extending protrusion in a lockable manner.

For example, in the first state, the activation button may be arranged such that the first locking surface of the first radially extending protrusion abuts the first locking interacting surface of the rear cap, and such that the second locking surface of the second radially extending protrusion abuts the second locking interacting surface of the delivery member cover. Thus, axial movement of the rear cap into its non-blocking position, and axial movement of the delivery member cover into its retracted position, is prevented by the activation button. In the second state, the activation button may be arranged such that the first locking surface is distant from the the first locking interacting surface of the rear cap by that the first radially extending protrusion axially aligns with the first indentation of the rear cap, and such that second radially extending protrusion axially aligns with the second indentation of the delivery member cover. Thus, axial movement of the rear cap into its non-blocking position, and axial movement of the delivery member cover into its retracted position, is enabled by the activation button, as the first radially extending protrusion is axially movable within the first indentation, and as the second radially extending protrusion is axially movable within the second indentation. According to at least alternative example embodiment, the lockable engagement between the activation button and the delivery member cover is omitted, and only the lockable engagement between the activation button and the rear cap, as described above, is present.

According to at least one example embodiment, the sub-assembly is configured such that the delivery member cover is movable into its retracted position is independently of the position of the activation button.

Hereby, the order, or sequence, of moving the delivery member cover into tis retracted position, and bringing the activation button into its second state, may vary.

According to at least one example embodiment, the sub-assembly further comprises a drive spring biasing the plunger rod towards the proximal end of the housing.

For example, the drive spring may be biased between a protrusion of the plunger rod, and the holding sleeve. Thus, the holding sleeve may be tubular and comprising a receiving recess in which the drive spring and plunger rod are arranged. The driver spring may preferably be compressed between a distal inner surface of the holding sleeve and the protrusion of the plunger rod.

According to a second aspect of the present invention a medicament delivery device for expelling medicament from a medicament container via a delivery member is provided. The medicament delivery device comprises a sub-assembly according to the first aspect of the invention.

Effects and features of the second aspect of the invention are largely analogous to those described above in connection with the first aspect of the invention. Embodiments mentioned in relation to the first aspect of the invention are largely compatible with the second aspect of the invention.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

FIG. 1 schematically shows a perspective view of an example of a medicament delivery device;

FIG. 2 shows an exploded view of the medicament delivery device in FIG. 1;

FIGS. 3A and 3B are perspective views of at least a part of a sub-assembly of the medicament delivery device in FIG. 2,

FIG. 4A is radial cross sectional view of a first sub-assembly with an activation button in a first state;

FIG. 4B is a perspective view of the first sub-assembly of FIG. 4A;

FIG. 4C is radial cross sectional view of the first sub-assembly of FIG. 4A with the activation button in a second state;

FIG. 5A is a perspective, partly cross sectional view of a second sub-assembly;

FIG. 5B is another perspective, partly cross sectional view of the second sub-assembly of FIG. 5A;

FIG. 6 is a perspective, partly cross sectional view of a third sub-assembly;

FIG. 7 is a perspective, partly cross sectional view of a fourth sub-assembly; and

FIG. 8 a perspective, partly cross sectional view of a fifth sub-assembly;

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

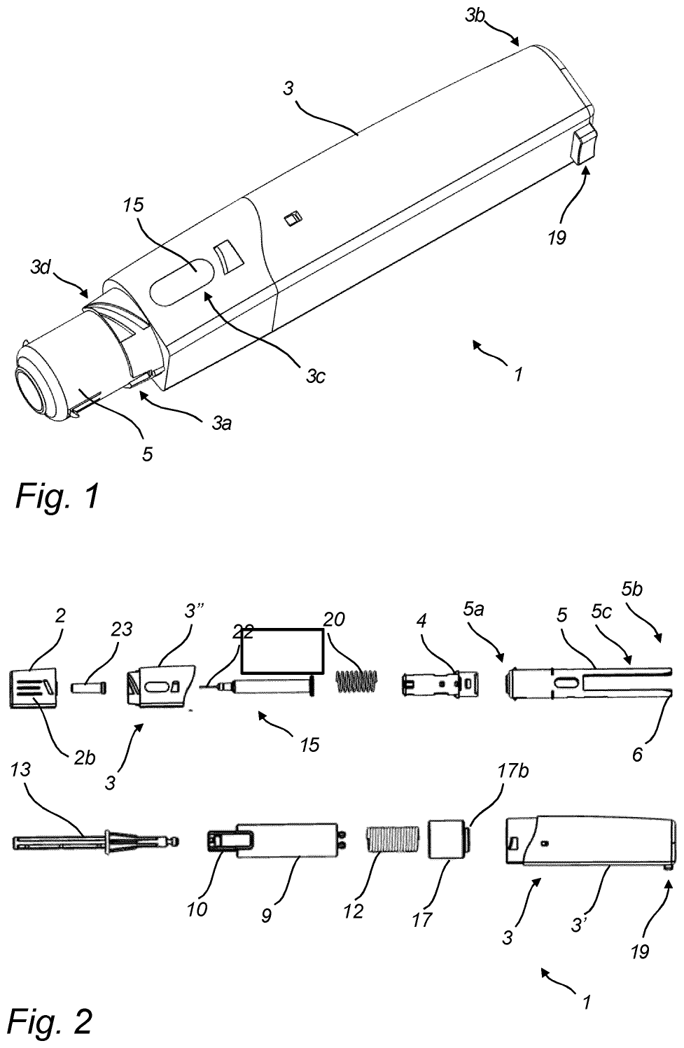

FIG. 1 shows an example of a medicament delivery device 1 configured to expel medicament from a medicament container 15 loaded in a housing 3 of the medicament delivery device 1. The housing 3 has a proximal end 3a and a distal end 3b, and an optional window c (or opening) arranged between the proximal end 3a the distal end 3b for visual inspection of the medicament container 15.

The medicament delivery device 1 comprises a delivery member cover 5. The delivery member cover 5 is arranged in the housing 3 and extends proximally from the proximal end 3a of the housing 3. The medicament delivery device 1 may further comprise a cap (shown in FIG. 2) which is removably arranged to the housing 3 (or alternatively to delivery member cover 5). Thus, in FIG. 1, the medicament delivery device 1 is shown in a state in which the cap has been removed, and the medicament delivery device is prepared for use.

The delivery member cover 5 is configured to be moved linearly, or axially, relative to the housing 3 from a protracted position as shown in FIG. 1, into a retracted position in which the delivery member cover 5 is received further into the housing 3. Typically, the delivery member cover 5 is biased in the proximal direction towards the protracted position.

The medicament delivery device 1 further comprises an activation button 19 configured to participate in the activation of discharge of medicament from the medicament container 15. Typically, also the movement of the delivery member cover 5 further into the housing 3 towards the retracted position participates in the activation of discharge of medicament from the medicament container 15, as will be further described below.

FIG. 2 shows an exploded view of the medicament delivery device 1. First, the general configuration of the medicament delivery device 1 will be described, then embodiments and certain aspects of various sub-assemblies 11, 111, 211, 311, 411 will be described in greater detail. The sub-assemblies of the medicament delivery device 1 may be defined by combining specific components of the medicament delivery device 1.

The medicament delivery device 1 comprises a medicament container carrier 4 extending along a longitudinal axis from a proximal end to a distal end. The medicament container carrier 4 may preferably be fixedly attached to the housing 3 of the medicament delivery device 1, e.g. by a snap lock or other conventional engagement means. Alternatively, the medicament container carrier 4 may form an integral part of the housing 3. The medicament container carrier 4 and the housing 3 are preferably made of the same material, e.g. plastic. The medicament container carrier 4 is configured to house and support the medicament container 15 inside the medicament delivery device 1.

The housing 3 of the medicament delivery device 1 is in FIG. 2 divided into two housing sub-portions, i.e. a first housing sub-portion 3′ and a second housing sub-portion 3″. The first housing sub-portion 3′ is arranged distally of the second housing sub-portion 3″. As an alternative, the first and second housing sub-portions 3′, 3″ may be made in one piece. Typically, reference will be made to the housing 3 in the description, and not to its housing sub-portions 3′, 3″.

The medicament delivery device 1 comprises the delivery member cover 5 described with reference to FIG. 1. The delivery member cover 5 comprises a proximal end 5a and a distal end 5b. The delivery member cover 5 is preferably tubular, or comprises at least a tubular portion. The delivery member cover 5 of FIG. 2 comprises a distal end portion 5c having a pushing surface 6. In the embodiment of FIG. 2, the distal end 5b comprises two distally extending legs, i.e. a first distally extending leg and a second distally extending leg. Thus, the pushing surface 6 may be comprised in either one of the two distally extending legs, or the pushing surface 6 is divided into a first pushing surface comprised in the first distally extending leg, and into a second pushing surface comprised in the second distally extending leg.

The medicament delivery device 1 comprises a cap 2. The cap 2 is removably arranged relative to the housing 3. For example, the cap 2 is removably arranged to the housing by means of a threaded structure. This may e.g. be achieved by a first threaded structure 3d (shown at the proximal end 3a of the housing 3 in FIG. 1), and a corresponding second threaded structure (not shown) comprised in the interior of the cap 2. In the embodiment of FIG. 2, the cap 2 comprises a gripping portion 2b and an internal structure for interacting with a shield of a delivery member, as further explained below. The gripping portion 2b comprises a gripping surface which a user may interact with in order to remove the cap 2 from the housing 3. For example, the user may twist the cap 2 whereby the cap 2 is threaded off the housing 3 by means of the threaded structure.

The medicament delivery device 1 comprises a plunger rod 13 operably arranged to, upon activation, move in the proximal direction. Thus, in the embodiment of FIG. 2, the plunger rod 13 is configured to move axially inside the housing 3, and inside the medicament container carrier 4. During a medicament delivery operation, the plunger rod 13 moves from a first axial position towards the medicament container 15 for expelling the medicament. The plunger rod 13 may have a cylindrical body.

The medicament delivery device 1 comprises a drive spring 12 configured to bias the plunger rod 13 in the proximal direction. Thus, the plunger rod 13 is pre-tensioned by the drive spring 12. In the embodiment of FIG. 2, the plunger rod 13 is axially fixed relative to the medicament container carrier 4 and the housing 3 in the first axial position, when the delivery member cover 5 is in the protracted position prior to a medicament delivery operation.

The medicament delivery device 1 comprises an activation button 19 operatively arranged to, upon activation, move from a first state to a second state, which will be described for the various sub-assemblies in the following.

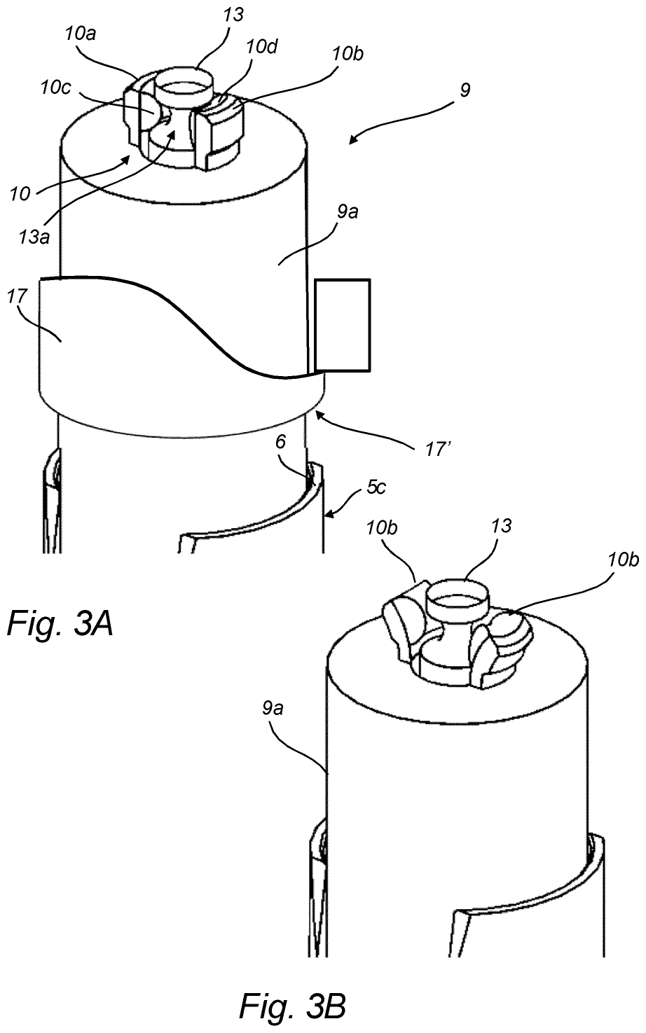

The medicament delivery device 1 comprises a holding sleeve 9 fixedly attached to the housing 3. The holding sleeve 9 is preferably tubular, or at least comprises a tubular portion (as the base portion 9a described below). The holding sleeve 9 comprises at least one holding arm 10 arranged to lock the plunger rod 13 in its pre-tensioned state. For example, each one of the holding arm(s) 10 comprises a protrusion, or hook, configured to interact with a corresponding indentation, or nob, of the plunger rod 13 (as shown in FIGS. 3A and 3B).

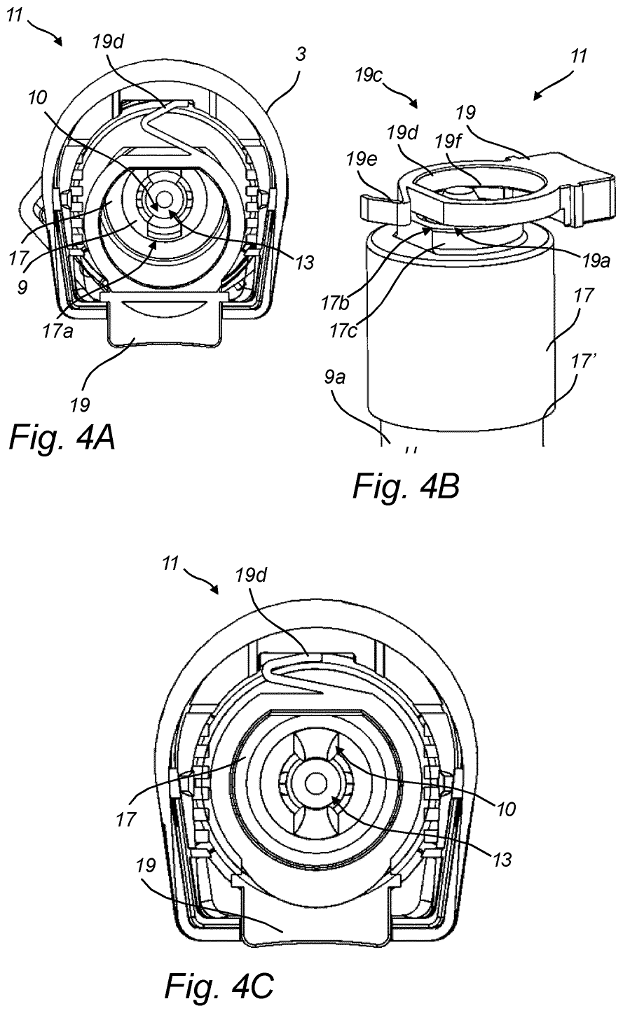

The medicament delivery device 1 comprises a rear cap 17 having a blocking surface 17a (better shown in FIGS. 4A and 4B) arranged to prevent the holding arm 10 from releasing the plunger rod 13 from its pre-tensioned state. The rear cap 17 is operatively configured to move distally from a blocking position, in which the blocking surface 17a is arranged to restrict movement of the holding arm 10, into a non-blocking position in which the blocking surface 17a is distant from the position restricting movement of the holding arm 10.

The medicament delivery device 1 comprises a distal resilient member 20. The distal resilient member 20 may for example be a spring, such as a coil spring. The distal resilient member 20 is configured to bias the delivery member cover 5 in the proximal direction. In the embodiment of FIG. 2, the distal resilient member 20 is configured to bias the delivery member cover 5 against the medicament container carrier 4.

The medicament delivery device 1 can be loaded with a medicament container 15. The medicament container 15 typically comprises a delivery member 22, such as a needle 22, and a ring needle shield, RNS, 23 arranged to protect the delivery member 22. The medicament container 15 my alternatively comprise an RNS needle cover and/or an RNS plastic cap (not shown). The cap 2 typically comprises an internal structure for interacting with the RNS 23. The internal structure may be configured to remove the RNS 23 upon cap removal. For embodiments in which the medicament container 15 is a syringe and the delivery member 22 is a needle, the medicament delivery device 15 may further comprise a syringes stopper and a syringe support (not shown).

In order to release the plunger rod 13, independent or dependent actions of the rear cap 17 and the activation button 19 are typically needed, which will be described in the following.

FIGS. 3A and 3B are perspective views of certain components of the medicament delivery device 1 of FIGS. 1 and 2. In each one of FIGS. 3A and 3B, the holding sleeve 9, the plunger rod 13, and the distal end portion 5c comprising the pushing surface 6 are shown. Moreover, in FIG. 3A, the rear cap 17 is shown in a partly cutaway view in order to better visualize the holding sleeve 9 and its interaction with the plunger rod 13.

The holding sleeve 9 comprises a base portion 9a from which at least one holding arm 10 distally extends. In the embodiment of FIGS. 3A and 3B, a first holding arm 10a, and a second holding arm 10b extends distally from the base portion 9a on opposite sides of the plunger rod 13. The first and second holding arms 10a, 10b are flexibly arranged relative to the base portion 9a. In more detail, each one of the holding arms 10a, 10b is arranged to move from an unflexed state in which the plunger rod is locked in its pre-tensioned state, as shown in FIG. 3A, to a flexed state in which the plunger rod 13 is released, as shown in FIG. 3B.

In the unflexed state of the first and second holding arms 10a, 10b, as shown in FIG. 3A, each one of the two holding arms 10a, 10b comprises a protrusion 10c, 10d extending radially inwards toward a central axis. Correspondingly, the plunger rod comprises a notch 13a arranged to receive the protrusions 10c, 10d of the first and second holding arms 10a, 10b. Thus, as the first and second holding arms 10a, 10b are kept in their unflexed state (e.g. by the rear cap 17 and/or the activation button 19), the plunger rod 13 will be kept in its pretensioned state owing to the engagement of the protrusions 10c, 1d of the first and second holding arms 10a, 10b with the notch 13a of the plunger rod 13.

As mentioned with reference to FIGS. 1 and 2, the delivery member cover 5 is configured to be brought into its retracted position by moving distally within the housing 3. During such movement, the pushing surface 6 abuts a proximal end portion 17′, such as a proximal sleeve portion 17′, of the rear cap 17. Hereby, the rear cap 17 may be moved from a blocking position of the holding arms 10 to a non-blocking position of the holding arms 10, described in greater detail below.

It should be mentioned that a different number of holding arms 10 may extend distally from the base portion 9a of the holding sleeve 9 than the two holding arms 10a, 10b shown in FIGS. 3A and 3B, why sometimes the general statement “at least one holding arm” or simply “the holding arm(s)” is used.

Typically, the rear cap 17 and/or the activation button 19 are configured to prevent the at least one holding arm 10 from releasing the plunger rod 13 from its pre-tensioned state by restricting movement of the at least one holding arm 10. That is, the rear cap 17 and/or the activation button 19 are configured to prevent the at least one holding arm 10 from moving from its unflexed state (in FIG. 3A) to its flexed state (in FIG. 3B). This will be described with reference to different sub-assemblies 11, 111, 211, 311, 411 in the following.

FIG. 4A shows a radial cross sectional view of a first sub-assembly 11 for a medicament delivery device, such as the medicament delivery device 1 of FIGS. 1 and 2, why the same reference numerals are used for like features without repeating a detailed descriptions thereof. FIG. 4B shows a perspective view of the sub-assembly 11 in which the housing 3 has been removed for increased clarity. The first sub-assembly 11 comprises a housing 3, a pre-tensioned plunger rod 13, a holding sleeve 9 fixedly attached to the housing 3 and comprising at least one holding arm 10 (as shown in FIGS. 3A and 3B), a rear cap 17 having a blocking surface 17a, and an activation button 19 having a locking surface 19a.

In FIGS. 4A and 4B, the holding arm(s) 10 of the holding sleeve 9 are prevented from moving to their unflexed state by that the blocking surface 17a of the rear cap 17 abuts the holding arm(s) 10. In the embodiment of FIGS. 4A and 4B, the rear cap 17 is tubular, and the blocking surface 17a is comprised in an inner rounded surface of the tubular rear cap 17, best shown in FIG. 4A. In more detail, and as shown in FIG. 4B, the rear cap 17 comprises a distal sleeve portion 17c, wherein the blocking surface 17a is comprised in an inner rounded surface of the distal sleeve portion 17c. Thus, in FIGS. 4A and 4B, the rear cap 17 is arranged in a blocking position in which the blocking surface 17a is arranged to prevent the holding arm(s) 10 from releasing the plunger rod 13 from its pre-tensioned state by abutting the holding arm(s) 10 and restricting movement thereof.

The rear cap 17 comprises a locking interacting surface 17b arranged to abut the locking surface 19a of the activation button 19 in a first state to thereby lock the rear cap 17 and preventing it from moving from the blocking position. Thus, the rear cap 17 cannot move further in the distal direction in the first state of the activation button. In more detail, in the embodiment of FIGS. 4A and 4B, the activation button 19 comprises an internal activation structure 19c arranged in the housing 3 and comprising a tubular activation portion 19d and an activation spring 19e biased against an inner surface of the housing 3 to arrange the activation button 19 in a pre-tensioned first state. The locking surface 19a is comprised in a proximally facing surface of the tubular activation portion 19d, and abuts the locking interacting surface 17b of the rear cap 17 in the first state. Thus, the rear-cap 17 is prevented from moving from its blocking position, regardless if the pushing surface of the delivery member cover 5 pushes a proximal end portion 17′ o the rear cap 17. In FIG. 4B it is shown that the locking interacting surface 17b is comprised in a distal facing surface of the distal sleeve portions 17c.

The activation button 19 is operatively arranged to, upon activation, move from the first state to a second state in which the locking surface 19a is displaced. This is shown in FIG. 4C. In more detail, by pressing the activation button 19, the tubular activation portion 19d is moved within the housing 3 by compressing the activation spring 19e. Hereby, the locking surface 19a will no longer be in contact with the locking interacting surface 17b of the rear cap 17, and the rear cap is thus allowed to move in the distal direction (e.g. by means of the delivery member cover 5 and the pushing surface 6 as described with reference to FIGS. 3A and 3B).

Stated differently, in the second state the locking interacting surface 17b is arranged distant from the locking surface 19a of the activation button 19. That is, in the second state of the activation button 19, the locking surface 19a is displaced to thereby allow the rear cap 17 to move distally and subsequently, allow the holding arm(s) 10 to move radially outwards to release the plunger rod 13. In more detail, the tubular activation portion 19d comprises a receiving recess 19f sized and dimensioned to receive at least the distal sleeve portion 17c of the rear cap 17. Hereby, the rear cap 17 is moved distally to a non-blocking position in which the blocking surface 17a is distant from the position restricting movement of the holding arm(s) 10, and thus no longer abut the holding arm(s) 10. Hereby, the holding arm(s) 10 is allowed to move radially outwards to release the plunger rod 13. As seen in FIGS. 4A-4C, the receiving recess 19f and the distal sleeve portion 17c are chamfered in a corresponding manner to facilitate the distal movement of the distal sleeve portion 17c into the receiving recess 19f.

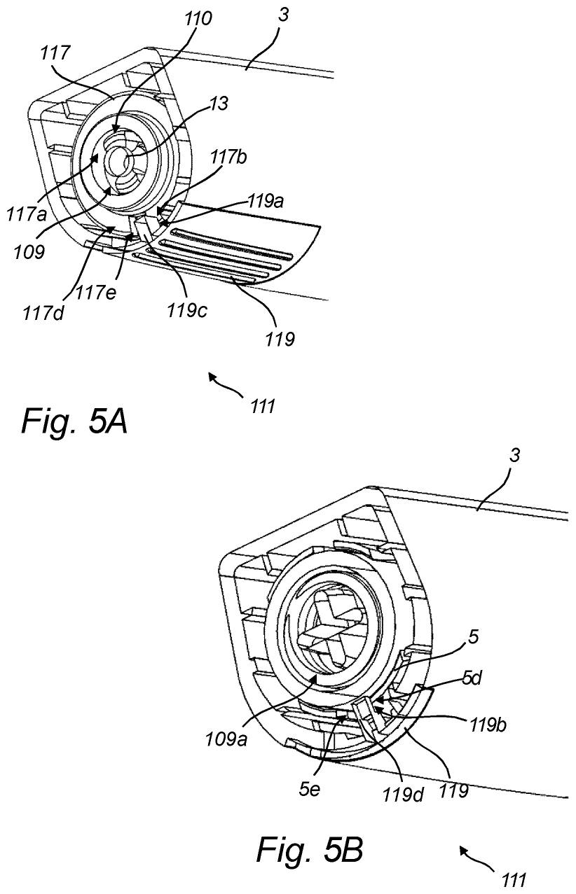

Turning to FIGS. 5A and 5B: these figures show an embodiment of a second sub-assembly 111 for a medicament delivery device configured to expel medicament from a medicament container 15. The second sub-assembly 111 of FIGS. 5A and 5B may replace the first sub-assembly 11 of FIGS. 4A-4C in the medicament delivery device 1 of FIGS. 1 and 2. The second sub-assembly 111 of FIGS. 5A and 5B is in large the same as the first sub-assembly 11 of FIGS. 4A-4C, with the differences described in the following. For like features, the same reference numerals are used as for the first sub-assembly 11 of FIGS. 4A-4C, without repeating a detailed descriptions thereof.

FIG. 5A shows a perspective, partly cross sectional view of the second sub-assembly 111 comprising a housing 3, a pre-tensioned plunger rod 13, a holding sleeve 109 fixedly attached to the housing 3 and comprising at least one holding arm 110 extending distally from a base portion 10a of the holding sleeve 109 (the holding sleeve 109 and the at least one holding arm 110 may be the same as the holding sleeve 9 and the at least one holding arm 10 as shown in FIGS. 3A and 3B), a rear cap 117 having a blocking surface 117a, and an activation button 119 having a locking surface 119a. The rear cap 117 typically comprises a distal sleeve portion wherein the blocking surface 117a is comprised in an inner rounded surface of the distal sleeve portion as described with reference to FIGS. 4A and 4B. In FIG. 5A, the rear cap 117 is arranged in its blocking position wherein the blocking surface 117a abuts the holding arm(s) 110 for preventing the holding arm(s) 110 from releasing the plunger rod 13 from its pre-tensioned state by restricting movement of the holding arm(s) 110, as previously described.

As shown in FIG. 5A, the activation button 119 comprises a first radially extending protrusion 119e comprising the locking surface 119a. The locking surface 119a may be a first locking surface 119a comprised in a proximal facing surface of the first radially extending protrusion 119c. Correspondingly, the rear cap 117 in FIG. 5A comprises a first structure 117d comprising a first locking interacting surface 117b and a first indentation 117e. The first structure 117d is engageable with the first radially extending protrusion 119e in a lockable manner. Stated differently, the first locking interacting surface 117b and the first indentation 117e form the first structure 117d. As long as the first radially extending protrusion 119e is unaligned with the first indentation 117e, the rear cap 117 is prevented from moving distally into a non-blocking position by the first radially extending protrusion 119c. Thus, the activation button 119 is arranged in its first state in which the first locking surface 119a is abutting the rear cap 117, here the first locking interacting surface 117b, to prevent the movement of the holding arm(s) 110 from releasing the plunger rod 13.

The activation button 119 is a slidable button, which upon activation rotates about a central axis of the sub-assembly 111. Thus, upon activation of the activation button 119, the first radially extending protrusion 119e is rotated relative to the rear cap 117. Hereby, the activation button 119 may be arranged in its second state in which the locking surface 119a is displaced to thereby allow the rear cap 117 to move distally and subsequently, allow the holding arm(s) 110 to move radially outwards to release the plunger rod 13. In more detail, in the second state of the activation button 119, the first radially extending protrusion 119e is axially aligned with the first indentation 117e, and the rear cap 117 is thus enabled to move distally into a non-blocking position as the first radially extending protrusion 119c runs within the first indentation 117e. Thus, in the second state, the blocking surface 117a no longer abuts the holding arm(s) 110.

The sub-assembly 111 may further comprise the delivery member cover 5 described with reference to FIG. 1, or at least be configured to interact with the delivery member cover 5 as described in the following. The activation button 19 may comprises a second radially extending protrusion 119d comprising a second locking surface 119b. Moreover, the delivery member cover 5 may comprise a second locking interacting surface 5d and a second indentation 5e, engageable with the second radially extending protrusion 119d in a lockable manner. As long as the second radially extending protrusion 119d is unaligned with the second indentation 5e, the delivery member cover 5 is prevented from moving distally inside the housing 5. Thus, in the first state of the activation button 119 the second locking surface 119b is abutting the delivery member cover 5, here the second locking interacting surface 5d, to prevent the movement of the delivery member cover 5. Thus, the delivery member cover 5 cannot move into its retracted position, and therefore cannot push the rear cap 17 into its non-blocking position.

Upon activation of the activation button 119 as described above, in addition to axially aligning the first radially extending protrusion 119c with the first indentation 117e, the second radially extending protrusion 119d is axially aligned with the second indentation 5e, and the delivery member cover 5 is thus enabled to move distally inside the housing 3, e.g. to push the rear cap 117 into its non-blocking position.

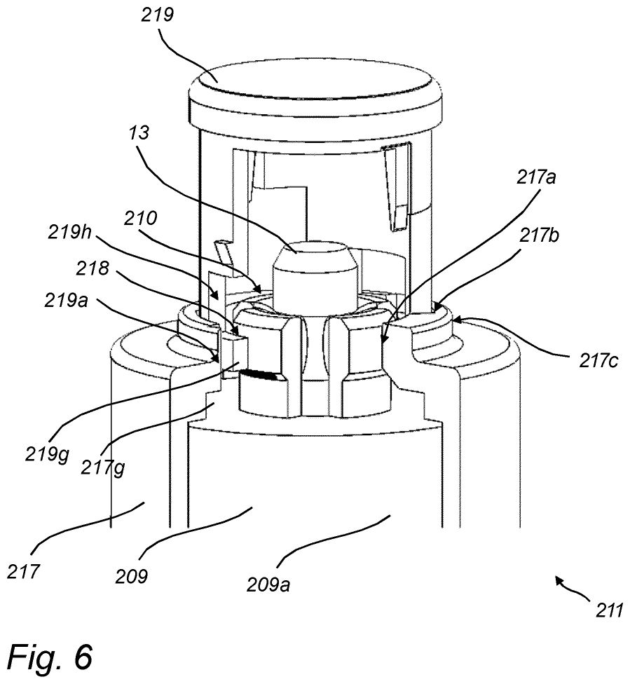

In the following, three different sub-assemblies 211, 311, 411 will be described with reference to FIGS. 6, 7 and 8. Any one of the sub-assemblies 211, 311, 411 of FIGS. 6, 7 and 8 may replace the first sub-assembly 11 of FIGS. 4A-4C in the medicament delivery device 1 of FIGS. 1 and 2. For like features, the same reference numerals are used as for the first sub-assembly 11 of FIGS. 4A-4C, without repeating a detailed descriptions thereof.

FIG. 6 shows a perspective, partly cross sectional view of third sub-assembly 211 comprising a housing (e.g. the housing 3 of FIGS. 1 and 3), a pre-tensioned plunger rod 13, a holding sleeve 209 fixedly attached to the housing and comprising four holding arms 110 extending distally from a base portion 209a of the holding sleeve 209. The holding sleeve 209 and the holding arms 210 may be the same as the holding sleeve 9 and the holding arms 10 as shown in FIGS. 3A and 3B, with the exception that that the holding sleeve 209 of the third sub-assembly 211 comprises four holding arms 210. The third sub-assembly 211 further comprises a rear cap 217 having a blocking surface 217a, and an activation button 219 having a locking surface 219a. The rear cap 217 typically comprises a distal sleeve portion 217c wherein the blocking surface 217a is comprised in an inner rounded surface of the distal sleeve portion 217c similar to that described with reference to FIGS. 4A and 4B. In FIG. 6, the rear cap 217 is arranged in its blocking position wherein the blocking surface 217a abuts two of the four holding arms 210 for preventing the corresponding holding arms from mowing radially outwards. The two holding arms which the blocking surface 217a abuts in the blocking position of the rear cap 217 may be referred to as a first sub-portion of holding arms 210.

In the third sub-assembly 211 of FIG. 6, the activation button 219 is tubular and comprises an inner facing surface 218 arranged to abut two of the four holding arms 210 for preventing the corresponding holding arms from mowing radially outwards. The two holding arms which the inner facing surface 218 abuts in the first state of the activation button 219 may be referred to as a second sub-portion of holding arms 210. Thus, the blocking surface 217a of the rear cap 217, and the inner facing surface 218 of the activation button 219 abuts different holding arms 210.

The rear cap 217 of the third sub-assembly 211 comprises an indentation 217b in which a portion of the activation button 219 is movable. Hereby independent axial movement of the activation button 219 and the rear cap 217 is achieved. Thus, the activation button 219 may be moved from its first state shown in FIG. 6, in which the locking surface 219a is abutting the rear cap 217, and the inner facing surface 218 abuts the second sub-portion of holding arms 210, to a second state in which the locking surface 119a is distant from the rear cap 217. For this purpose, described for one of the two holding arms in the second sub-portion of holding arms 210, the activation button 219 comprises a window 219h, and a proximal end portion 219g. As the activation button 219 is brought into its second state, the proximal end portion 219g is aligned with a cut-out 217g of the rear cap 217, and the window 219h is aligned with the corresponding holding arm to thereby allow the holding arm to move radially outwards. A similar configuration is typically included for the other one of the two holding arms in the second sub-portion of holding arms 210.

The rear cap 217 of the sub-assembly 211 is thus movable independently of the activation button 219, and may thus be moved axially in the distal direction from a blocking position in which the blocking surface 217a is arranged to prevent the holding arms of the first sub-portion of holding arms 210 from radially moving outwards, to a non-blocking position in which the blocking surface 217a is distant from the position restricting movement of the corresponding holding arms, in principal according to the same procedure as previously described, not repeated here again.

Thus, the release of the plunger rod 13 from its pre-tensioned state is achieved by moving the rear cap 217 into its non-blocking position, and the activation button 219 into its second state. However, the order, or sequence, in which the rear cap 217 is moved into its non-blocking position, and the activation button 219 is brought into its second state, does not matter.

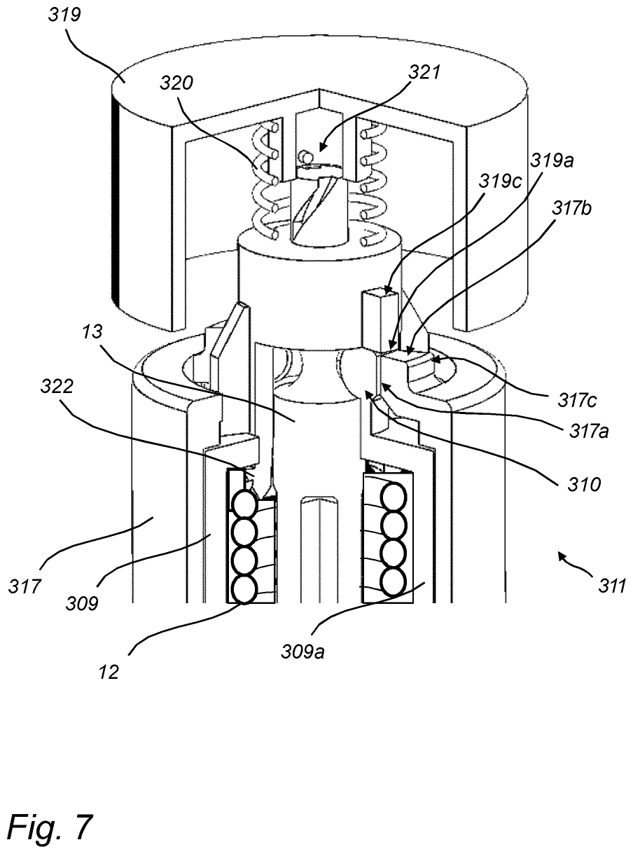

FIG. 7 shows a perspective, partly cross sectional view of a fourth sub-assembly 311 comprising a housing (e.g. the housing 3 of FIGS. 1 and 3), a pre-tensioned plunger rod 13, a drive spring 12, a holding sleeve 309 fixedly attached to the housing and comprising at least one holding arm 310 extending distally from a base portion 309a of the holding sleeve 309. The holding sleeve 309 and the holding arms 310 may be the same as the holding sleeve 9 and the holding arms 10 as shown in FIGS. 3A and B. The fourth sub-assembly 311 further comprises a rear cap 317 having a blocking surface 317a, and an activation button 319 having a locking surface 319a. The rear cap 317 typically comprises a distal sleeve portion 317c wherein the blocking surface 317a is comprised in an inner rounded surface of the distal sleeve portion 317c similar to that described with reference to FIGS. 4A and 4B. In FIG. 6, the rear cap 317 is arranged in its blocking position wherein the blocking surface 317a abuts the holding arms 310 for preventing the holding arms 310 from moving radially outwards.

As shown in FIG. 7, the activation button 319 comprises a radially extending protrusion 319c comprising the locking surface 319a. The locking surface 319a is in the embodiment of FIG. 7 comprised in a proximal facing surface of the radially extending protrusion 319c. Correspondingly, the rear cap 317 comprises a locking interacting surface 317b engageable with the radially extending protrusion 319c in a lockable manner. As long as the radially extending protrusion 319c is axially aligned with the locking interacting surface 317b of the rear cap 317, the rear cap 317 is prevented from moving distally into a non-blocking position.

In the embodiment of FIG. 7, the activation button 319 may be biased by means of an activation coil spring 320. The activation button 319 may further comprises a hooked shaped end portion 322 interacting with the base portion 309a of the holding sleeve 309, and arranged at the proximal end of the activation button 319. Thus, the activation button 319 may be compressed by means of the activation coil spring 320 and the hooked shaped end portion 322. Moreover, as the activation button 319 is compressed, and moved from its first state to its second state, a pin and thread arrangement 321 enables rotation of the radially extending protrusion 319c.

Thus, upon an axial applied force in the proximal direction the activation button 319 is activated, and the radially extending protrusion 319e rotates about a central axis of the sub-assembly 311 owing to the pin and thread arrangement 321. Hereby, the radially extending protrusion 319c is rotated relative to the rear cap 317. Thus, the activation button 319 may be brought into its second state in which the locking surface 319a is displaced to thereby allow the rear cap 317 to move distally and subsequently, allow the holding arm(s) 310 to move radially outwards to release the plunger rod 13. In more detail, in the second state of the activation button 319, the radially extending protrusion 319c is axially unaligned with the distal sleeve portion 317c, and the rear cap 317 is thus enabled to move distally into a non-blocking position as the radially extending protrusion 319c passes on a lateral side of the distal sleeve portion 317c. Thus, the blocking surface 317a no longer abut the holding arm(s) 310.

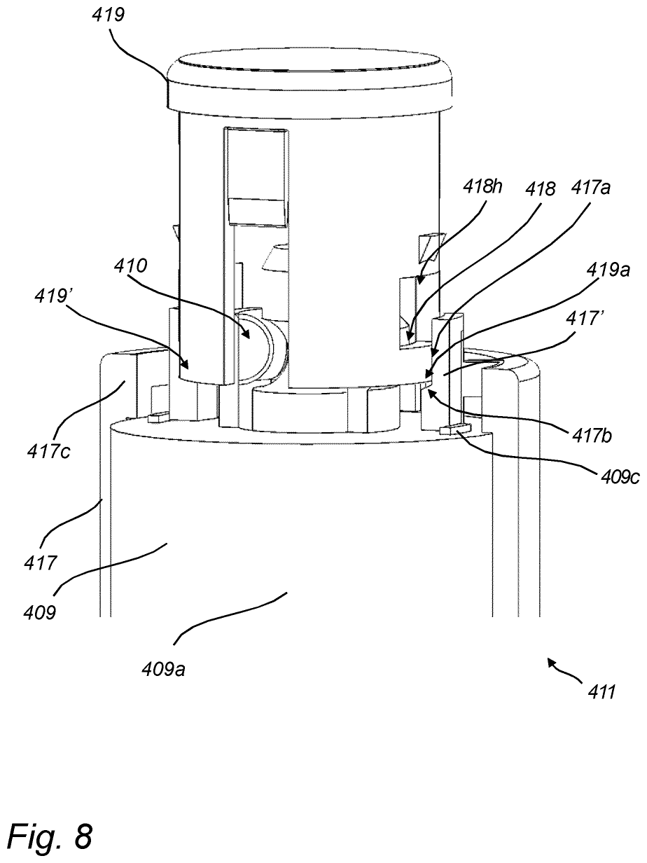

FIG. 8 shows a perspective, partly cross sectional view of fifth sub-assembly 411 comprising a housing (e.g. the housing 3 of FIGS. 1 and 3), a pre-tensioned plunger rod (e.g. the plunger rod 13 of FIGS. 1 and 3), a holding sleeve 409 fixedly attached to the housing and comprising at least one holding arm 410 extending distally from a base portion 409a of the holding sleeve 409. The holding sleeve 409 and the holding arms 410 may be the same as the holding sleeve 9 and the holding arms 10 as shown in FIGS. 3A and 3B. The fifth sub-assembly 411 further comprises a rear cap 417 having a blocking surface 417a, and an activation button 419 having a locking surface 419a. The rear cap 417 comprises at least one interaction leg 417c wherein the blocking surface 417a is comprised in an inner surface of the interaction leg 417c (facing a central axis of the sub-assembly 411). The interaction leg 417c is flexibly arranged relative to a sleeve portion 417c of the rear cap 417. In FIG. 8, the rear cap 417 is arranged in its blocking position wherein the blocking surface 417a abuts a proximal sleeve portion 419′ of the activation button 419 in order to prevent the holding arms 410 from mowing radially outwards. Thus, in FIG. 8, the blocking surface 417a is not in direct contact with the holding arms 410, but instead via the proximal sleeve portion 419′ of the activation button 419.

In the embodiment of FIG. 8, the holding sleeve 409 comprises a brim 409c preventing the interaction leg 417′ from flexing radially outwards.

The locking interacting surface 417b of the rear cap 417 is arranged to abut the locking surface 419a of the activation button 419 in the first state.

However, as the brim 409c is arranged to prevent the interaction leg 417′ from moving radially outwards, the activation button 419 cannot be moved in the proximal direction and thereby activate the release of the plunger rod 13. However, by first moving the rear cap 417 in the distal direction, a gap at least of the size of the brim 419c (i.e. in the axial direction) is achieved allowing the interaction leg 417′ to flex outwardly. This is achieved by pressing the activation button 419 in the proximal direction.

The activation button 419 is tubular and comprises an inner facing surface 418 arranged to abut the holding arms 410 for preventing the holding arms 410 from mowing radially outwards. The activation button 419 further comprises a window 418h for each holding arm 410 (of which only one window is shown in FIG. 8), the windows 418h being sized and dimensioned for receiving a corresponding holding arm 410.

The movement of the activation button 419 is thus dependent of a movement of the rear cap 417. Thus, subsequently to moving the interaction leg 417′ distally to an axial position above the brim 409c, the activation button 419 may be activated and thus moved from its first state to its second state. That is, as the rear cap 417 is moved axially in the distal direction from a blocking position in which the blocking surface 417a abuts a portion of the activation button 419 in order to prevent the holding arms 410 from radially moving outwards, to a non-blocking position in which the blocking surface 417a is distant from the position restricting movement of the corresponding holding arms, the activation button 419 may be moved proximally and push the interaction leg 417′ outwards, radially outside of the brim 409c. Thereafter the activation button 419 is brought into its second state by that the activation button 419 is moved proximally such that each window 418h aligns with a corresponding holding arm 410. Hereby, the holding arms 410 are enabled to move radially outwards to release the plunger rod 13 from its pre-tensioned state.

The inventive concept has mainly been described above with reference to a few examples. However, as is readily appreciated by a person skilled in the art, other embodiments than the ones disclosed above are equally possible within the scope of the inventive concept, as defined by the appended claims. For example, the plunger rod could for example be motor driven or manually driven instead of having an automatic spring-loaded structure.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

1-15. (canceled)

16. A sub-assembly for a medicament delivery device configured to expel medicament from a medicament container, the sub-assembly comprising:
a housing having a proximal end and a distal end;
a pre-tensioned plunger rod operably arranged to, upon activation, act on the medicament container for expelling a medicament;
a holding sleeve fixedly attached to the housing, the holding sleeve comprising at least one holding arm arranged to lock the plunger rod in its pre-tensioned state; and
a rear cap having a blocking surface arranged to prevent the holding arm from releasing the plunger rod from its pre-tensioned state by restricting movement of the holding arm, and being operatively configured to move distally from a blocking position to a non-blocking position in which the blocking surface is distant from the position restricting movement of the holding arm; and
an activation button having a locking surface, the activation button being operatively arranged to, upon activation, move from a first state in which the locking surface is abutting the rear cap to prevent the movement of the holding arm from releasing the plunger rod, to a second state in which the locking surface is displaced to thereby allow the holding arm to move radially outwards to release the plunger rod.

17. The sub-assembly according to claim 16, wherein the rear cap comprises a locking interacting surface arranged to abut the locking surface of the activation button in the first state to thereby lock the rear cap from moving to its non-blocking position.

18. The sub-assembly according to claim 17, wherein the locking interacting surface is arranged distant from the locking surface of the activation button in the second state to thereby enable the rear cap to move to its non-blocking position.

19. The sub-assembly according to claim 16, wherein the rear cap comprises an indentation in which a portion of the activation button is movable to thereby enable the rear cap to move distally from the blocking position to the non-blocking position independently of the activation button.

20. The sub-assembly according to claim 16, wherein the blocking surface of the rear cap is arranged to abut the holding arm of the holding sleeve in the blocking position.

21. The sub-assembly according to claim 16, wherein the blocking surface of the rear cap is arranged to abut at least a proximal sleeve portion of the activation button in the blocking position to prevent the holding arm to release the plunger rod from its pre-tensioned state by restricting movement of the holding arm.

22. The sub-assembly according to claim 16, wherein the activation button comprises an inner facing surface arranged to abut the holding arm of the holding sleeve in the first state.

23. The sub-assembly according to claim 16, wherein the holding sleeve comprises a base portion from which the holding arm distally extends, and wherein the holding arm is flexibly arranged relative to the base portion.

24. The sub-assembly according to claim 23, wherein the holding arm is arranged to move from an unflexed state in which the plunger rod is locked in its pre-tensioned state to a flexed state in which the plunger rod is released when the rear cap is arranged in its non-blocking position and the activation button is brought to its second state.

25. The sub-assembly according to claim 16, further comprising a delivery member cover arranged in the housing and extending proximally from the proximal end of the housing, the delivery member cover being configured to, during the medicament delivery operation, move linearly relative to the housing from a protracted position to a retracted position in which the delivery member cover is received further in the housing.

26. The sub-assembly according to claim 25, wherein the delivery member cover comprises a distal end portion having a pushing surface arranged to abut the rear cap when the delivery member cover is moved to its retracted position to thereby move the rear cap to its non-blocking position.

27. The sub-assembly according to claim 25, wherein the locking surface of the activation button is a first locking surface, and the activation button further comprises a second locking surface arranged distant to the first locking surface, wherein in the first state of the activation button, the second locking surface is abutting the delivery member cover to prevent the movement of the delivery member cover into its retracted position, and wherein in the second state of the activation button, the second locking surface is displaced to thereby allow the delivery member cover to move into its retracted position.

28. The sub-assembly according to claim 25, wherein the sub-assembly is configured such that the delivery member cover is movable into its retracted position independently of the position of the activation button.

29. The sub-assembly according to claim 16, further comprising a drive spring biasing the plunger rod towards the proximal end of the housing.

30. A medicament delivery device for expelling medicament from a medicament container via a delivery member, the medicament delivery device comprising the sub-assembly according to claim 16.
